(19) United States
(12) Patent Application Publication
(10) Pub. No.: US 2020/0015689 A1
(43) Pub. Date: Jan. 16, 2020

(54) WEARABLE BLOOD PRESSURE METER WITH ACTUATED CUFF

(71) Applicant: Verily Life Sciences LLC, South San Francisco, CA (US)

(72) Inventors: Michael Allen, San Francisco, CA (US); Shannon Fong, San Francisco, CA (US)

(21) Appl. No.: 16/508,203

(22) Filed: Jul. 10, 2019

Related U.S. Application Data
(60) Provisional application No. 62/697,903, filed on Jul. 13, 2018.

(51) Int. Cl. A61B 5/022

(52) U.S. Cl. CPC A61B 5/02241; A61B 5/02141; A61B 2562/164; A61B 2562/0247

(57) ABSTRACT

A wearable blood pressure meter includes a cuff, a tactile sensor, a cuff actuator, and a controller. The cuff is sized and shaped to wear around a body part having an artery. The tactile sensor is disposed on or adjacent to an inward facing surface of the cuff. The tactile sensor is positioned along the inward facing surface to align with the artery and to measure pressure applied to the tactile sensor by the artery when the cuff is worn around the body part. The cuff actuator is mechanically coupled to the cuff to cinch the cuff around the body part. The controller is coupled to the tactile sensor to record pressure signals from which a blood pressure measurement may be determined.

### CROSS-REFERENCE TO RELATED APPLICATIONS

This application claims the benefit of U.S. Provisional Application No. 62/697,903, filed Jul. 13, 2018, which is hereby incorporated by reference in its entirety.

### TECHNICAL FIELD

This disclosure relates generally to blood pressure metering, and in particular but not exclusively, relates to monitoring blood pressure at a digital artery.

### BACKGROUND INFORMATION

High blood pressure is a health concern for a large percentage of the population, but regular monitoring is not common-place. Blood pressure monitors are conventionally found in physician offices, hospitals, pharmacies, and occasionally in homes. However, those who suffer from high blood pressure may only occasionally monitor their blood pressure during a visit to the physician's office or while waiting for a prescription at the pharmacy. Additional monitoring of blood pressure is requested by many physicians, but patients may not follow through due to difficulty in obtaining readings, expense of portable units, or the associated discomfort while using the blood pressure monitor. The associated discomfort is typically due to the squeezing of the arm or wrist, for example. As such, it may be desirable to have portable, easy to use, and more comfortable painless blood pressure monitoring devices.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A is a perspective view illustration of wearable blood pressure meter worn on a finger, in accordance with an embodiment of the disclosure.

FIG. 1B is a functional block diagram illustrating functional components of a wearable blood pressure meter, in accordance with an embodiment of the disclosure.

FIGS. 2A and 2B illustrate various views of a finger-wearable blood pressure monitor, in accordance with an embodiment of the disclosure.

FIG. 3 is a perspective view illustration of a tactile sensor array, in accordance with an embodiment of the disclosure.

FIG. 4A is a perspective view illustration of interior components of a finger-wearable blood pressure monitor, in accordance with an embodiment of the disclosure.

FIG. 4B is a perspective view illustration of components of a cuff actuator, in accordance with an embodiment of the disclosure.

FIG. 4C is a top plan view illustration of interior components of a finger-wearable blood pressure monitor, in accordance with an embodiment of the disclosure.

FIG. 5 is a side view illustration of a finger-wearable blood pressure monitor having a reduced size finger cuff, in accordance with an embodiment of the disclosure.

### DETAILED DESCRIPTION

Embodiments of a system, apparatus, and method of operation for a wearable blood pressure meter are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

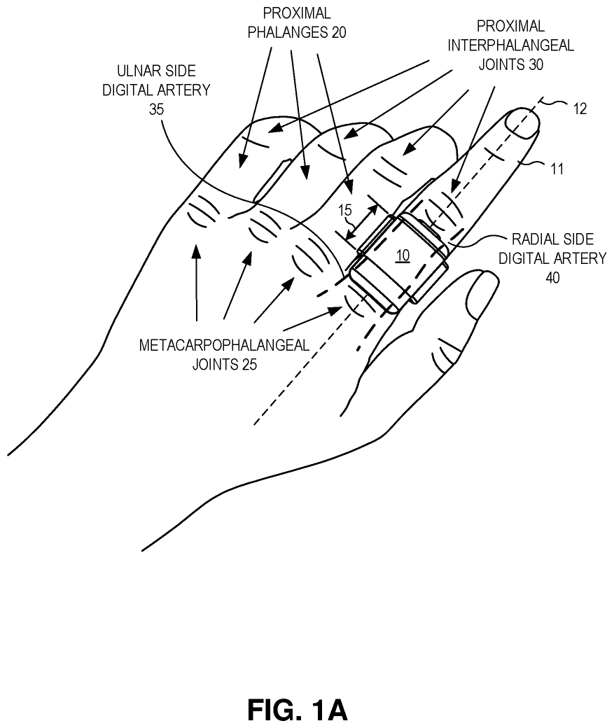

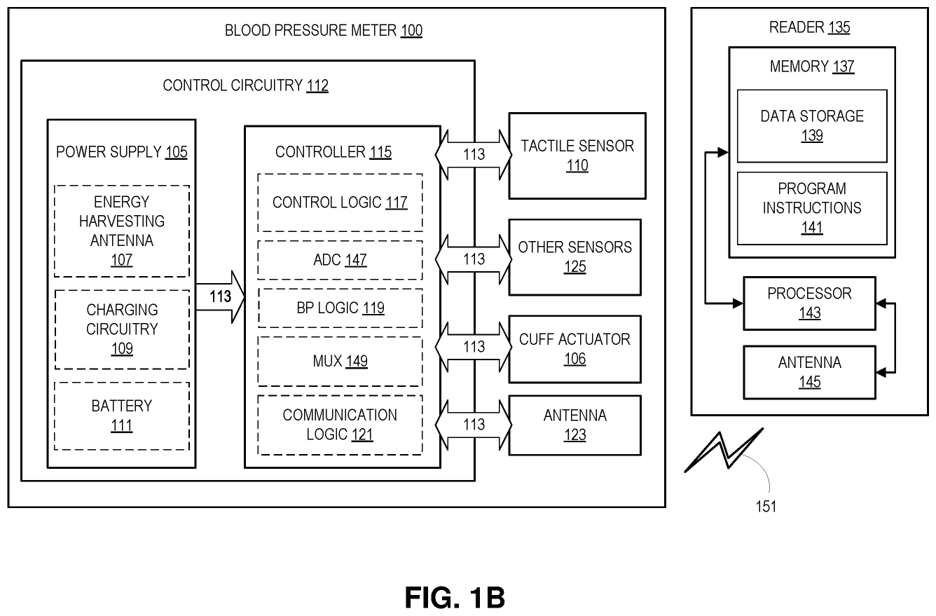

FIG. 1A is a perspective view illustration of wearable blood pressure meter 100 worn on a finger 11, in accordance with an embodiment of the disclosure. Blood pressure meter 10 is a non-intrusive and seamless way to meter the blood pressure of a wearer. Blood pressure meter 10 is a compact device that does not require bulky pumps, externally connected tubes or wires, or large batteries. As such, blood pressure meter 10 may be comfortably worn for extended periods to monitor and track a user's blood pressure while performing daily routines such as eating, sleeping, exercising, or otherwise. Accordingly, blood pressure meter 10 may also be referred to as a wearable blood pressure monitor.

FIG. 1A illustrates blood pressure meter 10 implemented as finger-wearable blood pressure monitor; however, it is contemplated that blood pressure meter 10 may be implemented in other form factors for sliding over and wearing on other extremities, such as wrists, arms, ankles, toes, or legs, or even as a neck band. Furthermore, although FIG. 1A illustrates blood pressure meter 10 positioned over a left-hand index finger, it may also be configured for use on a right-hand or other fingers or thumbs of a user.

Blood pressure meter 10 achieves its compact form factor, in part, due to its cuff actuator that cinches the cuff around a body part (illustrated as a finger cuff cinched around a finger) for occluding (or partially occluding) an artery within the body part. In one embodiment, the cuff actuator is implemented using a motor, gear reducing unit, and spool connected to a pulley system that draws in a pivoting section of the cuff towards a fixed section of the cuff thereby reducing the cross-sectional area defined by the cuff. In one embodiment, these cuff sections are rigid sections with the pivoting section including one or more links that pivot relative to the fixed section. The cuff and cuff actuator replace the need for a bladder and pump, thereby achieving a smaller form factor that is more energy efficient and capable of faster actuation relative to a pump and bladder mechanism.

The compact, power efficient nature of the cuff and cuff actuator of blood pressure meter 10 enables its form factor to be reduced such that the axial width 15 of the cuff itself fits over a proximal phalanx 20 of finger 11 between metacarpophalangeal joint 25 and proximal interphalangeal joint 30. This compact form factor provides the user freedom to bend and use finger 11 while wearing blood pressure meter 10, which lends itself well to longer term monitoring of blood pressure without significant user discomfort or disruption to daily activities. The configuration of blood pressure meter 10 illustrated in FIG. 1A obtains blood pressure measurements from either or both of ulnar side digital artery 35 or radial side digital artery 40 running in finger 11. Of course, not all advantages or features need be present in all embodiments.

FIG. 1B is a functional block diagram illustrating functional components of a wearable blood pressure meter 100, in accordance with an embodiment of the disclosure. Blood pressure meter 100 represents one possible implementation of blood pressure meter 10 illustrated in FIG. 1A. The illustrated embodiment of blood pressure meter 100 includes tactile sensor 110, control circuitry 112, other sensors 125, cuff actuator 106, and antenna 123. The illustrated embodiment of control circuitry 112 includes a power supply 105 and a controller 115. The illustrated embodiment of power supply 105 includes an energy harvesting antenna 107, charging circuitry 109, and a battery 111. The illustrated embodiment of controller 115 includes control logic 117, blood pressure (BP) logic 119, Analog-to-Digital Converter (ADC) 147, multiplexer (MUX) 149, and communication logic 121. Furthermore, as illustrated, the various components of blood pressure meter 100 are communicatively (e.g., electrically) coupled to each other via one or more interconnects 113.

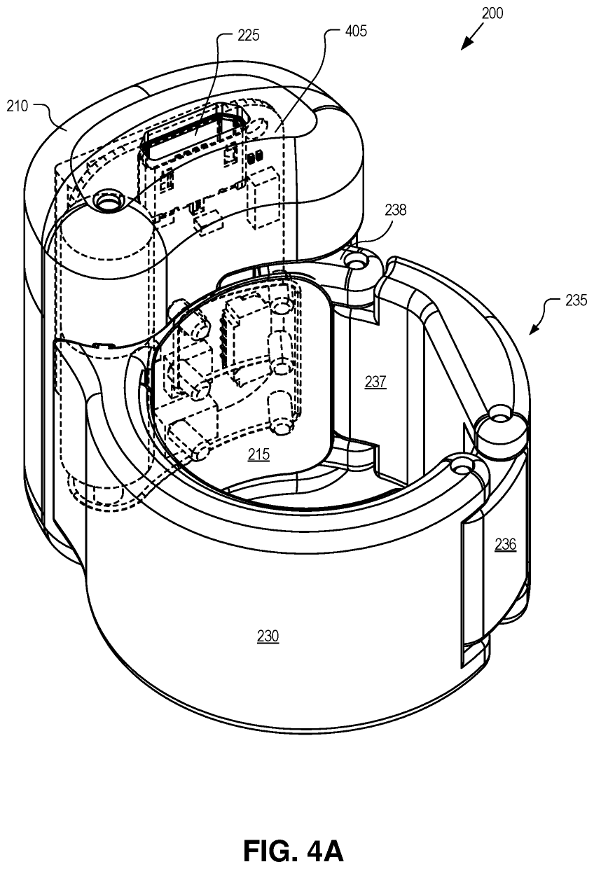

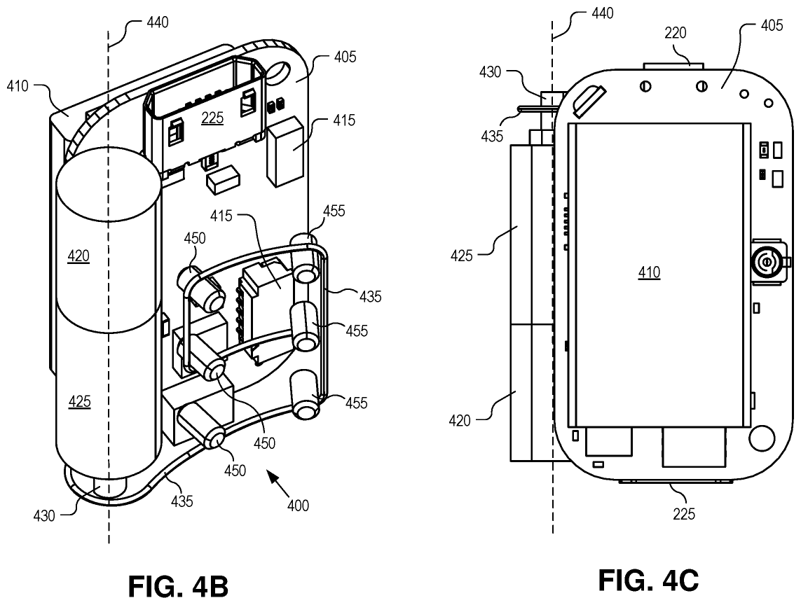

Power supply 105 supplies operating voltages to the controller 115 and various other sensors and components of blood pressure meter 100. Antenna 123 is operated by controller 115 to communicate information to and/or from blood pressure meter 100. In one embodiment, power supply 105, controller 115, and cuff actuator 106 are all mounted to a common substrate (e.g., substrate 405 illustrated in FIGS. 4A-C).

In the illustrated embodiment, power supply 105 includes battery 111 to power the various embedded electronics, including controller 115. Battery 111 may be inductively charged by charging circuitry 109 and energy harvesting antenna 107. In one embodiment, antenna 123 and energy harvesting antenna 107 are independent antennae, which serve their respective functions of energy harvesting and communications. In another embodiment, energy harvesting antenna 107 and antenna 123 are the same physical antenna that are time shared for their respective functions of inductive charging and wireless communications with reader 135. In yet other embodiments, battery 111 may be charged via a wire port of device 100.

Charging circuitry 109 may include a rectifier/regulator to condition the captured energy for charging battery 111 or directly power controller 115 without battery 111. Charging circuitry 109 may also include one or more energy storage devices to mitigate high frequency variations in energy harvesting antenna 107. For example, one or more energy storage devices (e.g., a capacitor, an inductor, etc.) can be connected to function as a low-pass filter.

Controller 115 contains logic to choreograph the operation of the other embedded components. Control logic 117 controls the general operation of blood pressure meter 100, including in some embodiments optionally providing a logical user interface, power control functionality, etc. Additionally, control logic 117 may control the actuation of cuff actuator 106 and receives and records pressure signals from a tactile sensor 110. ADC 147 may receive data from other sensors 125 and/or tactile sensor 110. ADC 147 may convert the received data to a digital format and provide the same to control logic 117 and/or BP logic 119. In some embodiments, ADC 147 may be coupled to tactile sensor 110 and the other sensors 125 via MUX 149, which controls the inflow of data to the ADC 147.

BP logic 119 may receive the measurements (e.g., capacitance measurements, etc.) from tactile sensor 110 and convert the measurements into equivalent pressure values. The pressure values may be in mmHg, for example. The pressure values may further be converted into pressure waveforms (e.g., a plurality of tactile waveforms) for each sensor element included in tactile sensor 110 that may be analyzed in either the time or frequency domains to determine mean arterial pressure, systolic blood pressure, and/or diastolic blood pressure at the digital artery. In some embodiments, the plurality of tactile waveform may be converted from a first waveform type (e.g., pressure at the digital artery) to a second waveform type (e.g., pressure at a brachial artery). BP logic 119 may analyze the plurality of tactile waveforms to determine arterial pulses for each of the plurality of tactile waveforms. The determined arterial pulses may subsequently be utilized to determine or estimate blood pressure.

Blood pressure meter 100 may use a variety of techniques such as oscillometry, auscultation, or applanation tonometry to estimate a user's blood pressure at an artery in an extremity (e.g., digital artery of a finger), which may subsequently be converted to a clinical or brachial blood pressure with a transfer function and/or a machine learning algorithm.

For applanation tonometry, cuff actuator 106 presses tactile sensor 110 into the body part over an artery, which may deform the artery. The artery may or may not be deformed to occlusion. As the pressure applied to tactile sensor 110 by the body part is slowly reduced, the artery may slowly convert back to a normal shape, and may pass through a point where the internal pressure equals the external pressure exerted on the artery by tactile sensor 110. This point may occur when a local radius of the artery approaches infinity (i.e., flattens), at least in reference to a size of a sensor element on tactile sensor 110. In this state, e.g., with the local region of the artery being flat, the blood flow variations in the artery due to heart beats may cause the flat area of the artery to experience pressure fluctuations (e.g., arterial pulses). A maximum fluctuation, representing one of the arterial pulses having a pulse amplitude larger than the pulse amplitude of any other one of the arterial pulses, may occur at the flat condition. The pressure fluctuations may decrease when the local region is not quite flat. In some embodiments, the arterial pulse having a pulse amplitude greater than the pulse amplitude of any other arterial pulse included in all of the plurality of tactile waveforms is known as a basis arterial pulse. While the above operation was discussed in terms of a controlled reduction in pressure by cuff actuator 106 applied between a body part and tactile sensor 110, the operation may alternatively be performed using a controlled increase in pressure and the pressure changes may be measured during the controlled increase.

In some embodiments, BP logic 119 may receive sound recordings from a microphone to implement auscultatory blood pressure estimation. The microphone may be part of other sensors 125, which may be arranged to record blood pulses occurring in the artery. BP logic 119 may analyze the sound recordings in relation to pressure data received from tactile sensor 110 to determine a pressure when Korotkoff sounds begin and end. If the pressure is decreasing during this time from an occluded state of the artery, the pressure corresponding to the beginning of the Korotkoff sounds may be an estimate of the systolic blood pressure, whereas the pressure corresponding to the ending of the Korotkoff sounds may be an estimate of the diastolic blood pressure.

In some embodiments, BP logic 119 may determine the mean arterial pressure (MAP), systolic blood pressure (SBP), and diastolic blood pressure (DBP) using oscillometry. The determination of the mean arterial pressure, systolic blood pressure, and diastolic blood pressure may be similar to applanation tonometry techniques. For example, the pressure signals from tactile sensor 110 may measure pressure changes due to blood flow in the digital artery. The pressure oscillations may start small, increase to a maximum amplitude, and reduce. Similar to the applanation tonometry technique, the applied pressure at maximum amplitude may be an estimate of the mean arterial pressure. From the measured pressure oscillations BP logic 119 may determine the mean arterial pressure, the systolic blood pressure, and the diastolic pressure. The systolic blood pressure and diastolic blood pressure may be calculated from the measured mean arterial pressure through one or more regressions (e.g., linear regression).

In some embodiments, BP logic 119 may perform BP estimations using all three techniques. The BP estimations from the three different techniques may then be compared to determine a closest estimation of the user's BP at the peripheral artery in the extremity. Additionally, or alternatively, BP logic 119 may utilize the blood pressure estimates from the oscillometry and auscultatory techniques as reference data to confirm and/or verify the accuracy of the blood pressure estimate from tactile sensor 110 determined with regularized regression modeling or a machine learning algorithm.

Control logic 117 may receive diagnostic data from other sensors 106, which may include a temperature sensor, accelerometer, photoplethysmograph (PPG), and microphone. The data may be analyzed to determine if any of the measurements are outside of established thresholds and, if so, respond accordingly. For example, if accelerometer data shows that the body part was moving more than desired during a blood pressure reading, control logic 117 may reject that reading. Additionally, control logic 117 may determine the user's heart rate (HR), respiratory rate (RR), and/or oxygen saturation (SpO2) based on PPG sensor data. Lastly, temperature data may be used to adjust any blood pressure estimations if the temperature is outside of an established range.

Communication logic 121 provides communication protocols for wireless communication with reader 135 via antenna 123. In one embodiment, communication logic 121 provides backscatter communication via antenna 123 when in the presence of an electromagnetic field 151 output from reader 135. In one embodiment, communication logic 121 operates as a smart wireless radio-frequency identification (“RFID”) tag that modulates the impedance of antenna 123 for backscatter wireless communications. The various logic modules of controller 115 may be implemented in software/firmware executed on a general purpose microprocessor, in hardware (e.g., application specific integrated circuit), or a combination of both. Of course, communication logic 121 and antenna 123 may implement other communication standards, such as WiFi, Bluetooth, etc.

The illustrated embodiment also includes reader 135 with a processor 143, an antenna 145, and memory 137. Memory 137 includes data storage 139 and program instructions 141. As shown reader 135 may be disposed outside of device 100, but may be placed in its proximity to charge device 100, send instructions to device 100, and/or extract data from device 100. In one embodiment, reader 135 may resemble a hand held portable device that provides a holder or case for device 100. In one embodiment, reader 135 may represent a portable computing device, such as a smartphone, a tablet, a laptop, or otherwise.

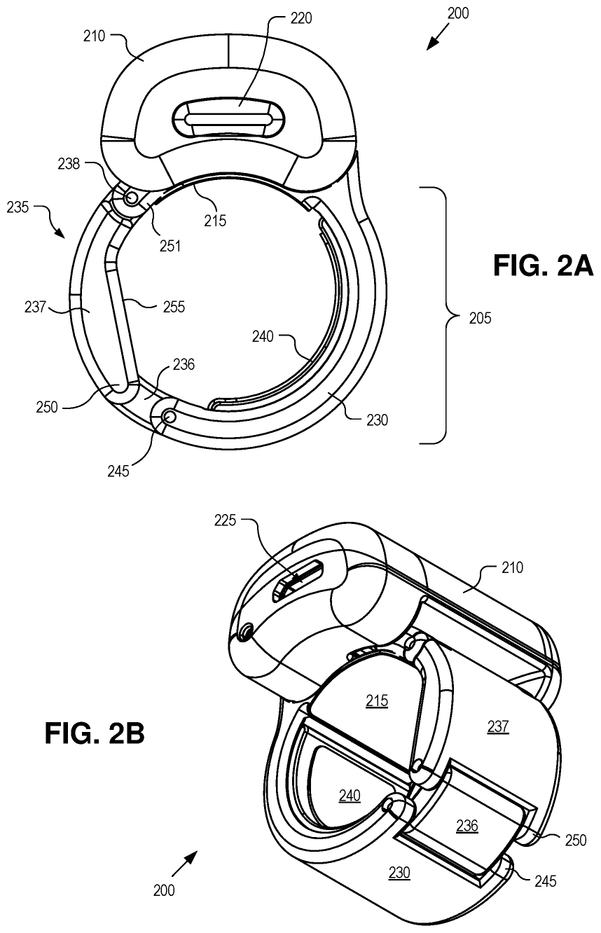

FIGS. 2A and 2B illustrate various views of a finger-wearable blood pressure monitor 200, in accordance with an embodiment of the disclosure. Monitor 200 is one possible finger-wearable implementation of wearable blood pressure meters 10 or 100. The illustrated embodiment of monitor 200 includes a cuff 205, a housing 210, a flexible cover guard 215, a button 220, and a data/power port 225. The illustrated embodiment of cuff 205 includes a fixed section 230, a pivoting section 235, and an inward facing surface 240 in or on which a tactile sensor array is disposed. FIGS. 4A-C illustrate the various internal components of monitor 200, which include a cuff actuator 400, a substrate 405, a battery 410, and control circuitry 415. In some embodiments, a portion of control circuitry 415 may also be disposed within fixed section 230 of cuff 205. Operation of the internal components of monitor 200 are discussed in greater detail below.

Cuff 205 is sized and shaped to slide over and wear around a body part, such as finger 11. A tactile sensor array is disposed on or adjacent to inward facing surface 240 and positioned angularly within cuff 205 to align with (e.g., overlap) an artery. When cuff 205 is constricted or otherwise cinched, the tactile sensor array is pressed into the artery to measure blood pressure fluctuations in the artery. For example, in the illustrated embodiment, the tactile sensor array is disposed in or on the curvature of fixed section 230 and aligns to the ulnar side digital artery 35. In one embodiment, the tactile sensor array is centered at approximately 120 degrees from top dead center of cuff 205.

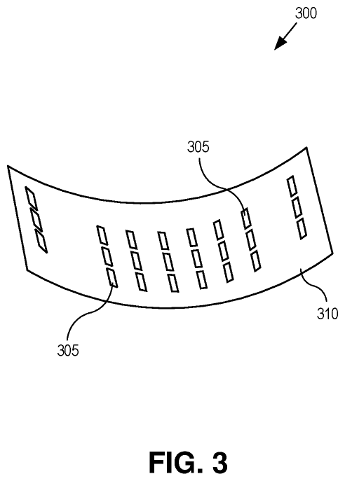

FIG. 3 is a perspective view illustration of an example tactile sensor array 300, in accordance with an embodiment of the disclosure. Tactile sensor array 300 is one possible implementation of tactile sensor 110. Tactile sensor array 300 includes deformable sensor elements 305 organized into rows and columns along a curved surface 310 that aligns with and overlaps an artery. In one embodiment, curved surface 310 conforms to inward facing surface 240. In one embodiment, sensor elements 305 are capacitive sensor elements, though other types of sensors may be implemented. Capacitive sensor elements 305 deform due to fluctuations in the arterial wall caused by the blood pressure fluctuations. These fluctuations may change a shape, e.g., height, of one or more deformable capacitive sensor 305, which in turn changes their capacitance values. The changing capacitance is measured, which provides an indication of the blood pressure in the digital artery. The capacitance levels of the capacitive sensors may be converted into pressure levels, e.g., mmHg, via a factory calibration procedure, and forms tactile waveforms. Each of the tactile waveforms corresponds to the pressure applied to a respective one of the plurality of sensors over a period of time. Features of the tactile waveforms may be used to estimate a mean arterial pressure, a systolic blood pressure, and a diastolic blood pressure. Although FIG. 3 illustrates an array implementation of tactile sensor 110, in other embodiments, tactile sensor 110 may be implemented with a single sensor element. The use of an array of sensor elements 305 alleviates the requirement of precise placement of cuff 205 and device performance across users with differing anatomy can be improved.

Returning to the embodiment illustrated in FIGS. 2A and 2B, the tactile sensor array is disposed along inward facing surface 240 of fixed section 230. Fixed section 230 is rigidly mounted to housing 210. Pivoting section 235 includes a proximal end having a pivot joint 245 that couples to fixed section 230 and a distal end that is drawn into an opening in housing 210 above flexible cover guard 215. Flexible cover guard 215 is attached to the underside of housing 210 and defines an upper portion of cuff 205 while also protecting the internal components of housing 210. The distal end of pivoting section 235 is mechanically connected to the cuff actuator within housing 210 and drawn into housing 210 when cinching around finger 11, thereby reducing the cross-sectional area defined by cuff 205.

In the illustrated embodiment, pivoting section 235 includes three links 236, 237, and 238 interconnected by pivot joints 250 and 251. Links 236 and 237 may be interchangeable with different sizes of links to accommodate different sizes of body parts (e.g., fingers) for different users. For example, three different sizes of links representing small, medium, and large may be provided. Links 236, 237, and 238 while pivoting relative to each other may otherwise be fabricated of structurally rigid material.

In the illustrated embodiment, pivoting section 235 further includes a flat portion 255 oriented in an opposing position (e.g., 120 degrees from top dead center of housing 210 in the opposite direction) to the tactile sensor array. Flat portion 255 is angularly positioned within cuff 205 to press against the opposing side digital artery (e.g., radial side digital artery 40 in FIG. 1A) when cuff 205 is cinched. Flat portion 255 targets compression of the opposing side digital artery, which enhances blood pressure measurements at the tactile sensor array on the other side of finger 11. Pressure waves from cyclical occlusion of the opposing side digital artery are believed to propagate to the tactile sensor array through the digital arteries, further enhancing measurement sensitivity.

Referring to FIGS. 4A-C, the components internal to housing 210 are illustrated. The internal components include cuff actuator 400, substrate 405, battery 410, and control circuitry 415. Battery 410 is mounted to the topside of substrate 405 while cuff actuator 400 and control circuitry 415 are mounted to the bottom side of substrate 405. The illustrated embodiment of cuff actuator 400 includes a motor 420, a gear reduction unit 425, a spool 430, a cord 435, and a pulley system. Motor 420 and gear reduction unit 425 are mounted to substrate 405. In one embodiment, gear reduction unit 425 provides a 700:1 gear reduction. Motor 420, gear reduction unit 425, and spool 430 are disposed along, and otherwise share, a common rotational axis 440, which when finger-wearable blood pressure monitor 200 is worn on finger 11 aligns substantially parallel to the longitudinal axis 12 of finger 11. This parallel configuration of motor 420, gear reduction unit 425, and spool 430 aids the compact form factor of finger-wearable blood pressure monitor 200 having an axial width of cuff 205 capable to fitting between metacarpophalangeal joint 25 and proximal interphalangeal joint 30.

The pulley system includes pulleys 450 mounted to substrate 405 and pulleys 455 mounted to the distal end (i.e., link 238) of pivoting section 235 of cuff 205. Cord 435 winds around spool 430, laces around pulleys 450 and 455 and terminates with a mechanical connection to the distal end of pivoting section 235. When cord 435 is wound around spool 430 by motor 420 and gear reduction unit 425, the lacing configuration of cord 435 around pulleys 450 and 445 provides further mechanical advantage for drawing the distal end of pivoting section 235 towards fixed section 230 and into housing 210. Cord 435 may be fabricated of a number of different materials and assume a variety of different form factors. For example, cord 435 may be implemented as a rope, a cable, a belt, a chain, or otherwise. In one embodiment cord 435 is fabricated of Kevlar lace, though other materials may be used. Furthermore, the diameter of spool 430 and the number pulleys 450 and 455 may be selected to adjust coiling speed and available torque.

Housing 210 further includes button 220 and port 225. In the illustrated embodiment, when finger-wearable blood pressure monitor 200 is slid over finger 11, button 220 faces toward the fingertip. Button 220 may provide a manual start feature for a user triggered blood pressure reading. Button 220 may also provide a manual stop feature enabling a user to terminate a blood pressure reading mid-cycle, for example in the event of discomfort while cuff 205 is cinched by cuff actuator 400. Alternatively, controller 415 may automatically obtain blood pressure readings according to a preprogrammed schedule and store the readings internally until they can be offloaded (e.g., to reader 135, to the cloud, etc.). Port 225 may be provided for charging and/or data communications. In one embodiment, port 225 is a micro USB port, though other port types may be implemented. In various embodiments, charging and/or data communications may be implemented wirelessly, over a wire, a mixture of the two, or both.

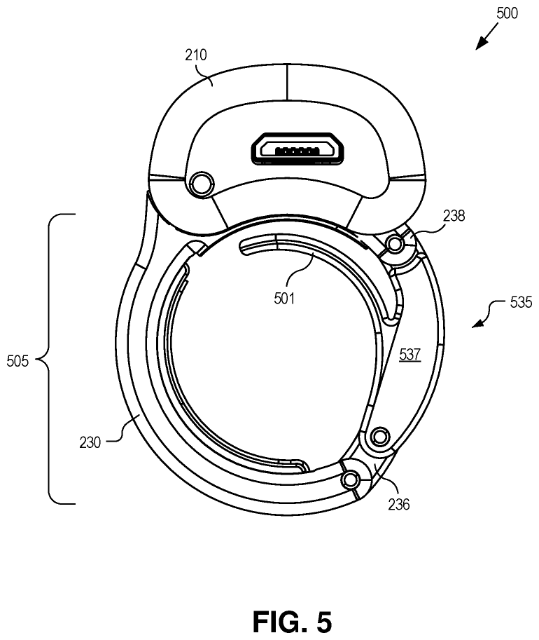

FIG. 5 is a side view illustration of a finger-wearable blood pressure monitor 500 having a reduced size finger cuff 505, in accordance with an embodiment of the disclosure. As illustrated, the middle link of pivoting section 535 has been swapped for a smaller link 537 having a spline 501 that further reduces the diameter of cuff 505 to accommodate smaller body parts (e.g., smaller fingers). As mentioned above, link 537 and 236 may be interchangeable with other sized links to accommodate fingers or body parts of variable sizes between different users.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A wearable blood pressure meter, comprising:
a cuff sized and shaped to wear around a body part having an artery;
a tactile sensor disposed on or adjacent to an inward facing surface of the cuff, the tactile sensor positioned along the inward facing surface to align with the artery and to measure pressure applied to the tactile sensor by the artery when the cuff is worn around the body part;
a cuff actuator mechanically coupled to the cuff to cinch the cuff around the body part; and
a controller coupled to the tactile sensor to record pressure signals from which a blood pressure measurement may be determined.

2. The wearable blood pressure meter of claim 1, wherein the cuff comprises:
a first section in or on which the tactile sensor is disposed; and
a pivoting section that pivots relative to the first section, wherein the cuff actuator is mechanically coupled to the pivoting section to draw in the pivoting section towards the first section to reduce a cross-sectional area defined by the cuff.

3. The wearable blood pressure meter of claim 2, wherein the wearable blood pressure meter comprises a finger-wearable blood pressure monitor and the cuff is sized and shaped to slide over a proximal phalanx of a finger.

4. The wearable blood pressure meter of claim 3, wherein the cuff has an axial width that permits a proximal interphalangeal joint and a metacarpophalangeal joint of the finger to bend while wearing the finger-wearable blood pressure monitor.

5. The wearable blood pressure meter of claim 2, wherein the first section comprises a fixed section that is curved, the tactile sensor conforms to a curvature of the fixed section, and the pivoting section includes a flat portion.

6. The wearable blood pressure meter of claim 5, wherein the tactile sensor is positioned angularly within the cuff on the fixed section to press against a first side digital artery and the flat portion of the pivoting section is positioned angularly within the cuff to press against a second side digital artery when the wearable blood pressure meter is worn on the body part.

7. The wearable blood pressure meter of claim 2, wherein the pivoting section includes two links with a first pivot joint connecting the two links and a second pivot joint connecting the pivoting section to the first section.

8. The wearable blood pressure meter of claim 7, wherein one or both of the two links are interchangeable with different sizes of links to accommodate different sizes of the body part for different users.

9. The wearable blood pressure meter of claim 2, wherein the cuff actuator comprises:
a motor disposed within a housing attached to the first section of the cuff;
a spool mechanically coupled to an output of the motor;
a pulley system; and
a cord extending from the spool, lacing around the pulley system, and attaching to a distal end of the pivoting section, wherein the cuff actuator cinches the cuff around the body part by winding the cord around the spool.

10. The wearable blood pressure meter of claim 9, wherein the cuff actuator further comprises a gear reduction unit disposed between the motor and the spool, wherein a common rotational axis of the motor, the gear reduction unit, and the spool aligns substantially parallel to a longitudinal axis of the body part when the wearable blood pressure meter is worn over the body part.

11. The wearable blood pressure meter of claim 9, wherein the pulley system comprises:
first pulleys mounted within the housing; and
second pulleys mounted to the distal end of the pivoting section of the cuff, wherein the cord is laced around the first and second pulleys to draw the second pulleys along with the distal end of the pivoting section towards the first section of the cuff when winding the cord around the spool.

12. The wearable blood pressure meter of claim 9, further comprising:
a flexible cover guard attached to the housing and defining a portion of the cuff, wherein the distal end of the pivoting section slides over the flexible cover guard into the housing when the cuff is cinched by the cuff actuator.

13. A finger-wearable blood pressure monitor, comprising:
a finger cuff sized and shaped to wear around a proximal phalanx of a finger, the finger cuff including a fixed section and a pivoting section that pivots relative to the fixed section;
a tactile sensor disposed on or adjacent to an inward facing surface of the finger cuff to measure pressure applied to the tactile sensor by an artery within the finger when the finger cuff is worn;
a cuff actuator mechanically coupled to the finger cuff to cinch the finger cuff around the finger thereby reducing a cross-sectional area defined by the finger cuff; and
a controller coupled to the tactile sensor to record pressure signals from which a blood pressure measurement may be determined.

14. The finger-wearable blood pressure monitor of claim 13, further comprising:
a housing, in which the cuff actuator and the controller are mounted, and to which the fixed section of the finger cuff is rigidly attached,
wherein a proximal end of the pivoting section pivotally attaches to the fixed section and a distal end of the pivoting section is drawn into an opening in the housing by a mechanical connection to the cuff actuator when cinching the finger cuff.

15. The finger-wearable blood pressure monitor of claim 14, wherein the cuff actuator comprises:
a motor;
a spool mechanically coupled to an output of the motor;
a pulley system; and
a cord extending from the spool, lacing around the pulley system, and attaching to the distal end of the pivoting section, wherein the cuff actuator cinches the finger cuff around the finger by winding the cord around the spool.

16. The finger-wearable blood pressure monitor of claim 15, wherein the cuff actuator further comprises a gear reduction unit disposed between the motor and the spool, wherein a common rotational axis of the motor, the gear reduction unit, and the spool aligns substantially parallel to a longitudinal axis of the finger when the finger-wearable blood pressure monitor is worn.

17. The finger-wearable blood pressure monitor of claim 15, wherein the pulley system comprises:
first pulleys mounted within the housing; and
second pulleys mounted to the distal end of the pivoting section of the finger cuff, wherein the cord is laced around the first and second pulleys to draw the second pulleys along with the distal end of the pivoting section towards the fixed section when winding the cord around the spool.

18. The finger-wearable blood pressure monitor of claim 13, wherein the fixed section is curved, the tactile sensors conforms to a curvature of the fixed section, and the pivoting section includes a flat portion.

19. The finger-wearable blood pressure monitor of claim 18, wherein the tactile sensor is positioned angularly within the finger cuff on the fixed section to press against a first side digital artery and the flat portion of the pivoting section is positioned angularly within the finger cuff to press against a second side digital artery when the finger-wearable blood pressure monitor is worn.

20. The finger-wearable blood pressure monitor of claim 13, wherein the pivoting section includes two links with a first pivot joint connecting the two links and a second pivot joint connecting the pivoting section to the fixed section.

21. The finger-wearable blood pressure monitor of claim 13, wherein the tactile sensor comprises an array of capacitive tactile sensor elements.
